(19) United States
(12) Patent Application Publication
(10) Pub. No.: US 2012/0284809 A1
(43) Pub. Date: Nov. 8, 2012

(54) KNOCK DOWN MODEL OF DICKKOPF HOMOLOGUE 3 (Dkk3) FOR ASSESSING ROLE OF SAID Dkk3 IN SPERMATOGENESIS AND SEX REVERSAL

(75) Inventors: Subeer S. Majumdar, New Delhi (IN); Deepika Sharma, Delhi (IN); Neerja Wadhwa, New Delhi (IN)

(73) Assignee: National Institute of Immunology, Gurgaon (IN)

(21) Appl. No.: 13/102,682

(22) Filed: May 6, 2011

(51) Int. Cl. A01K 67/027

(52) U.S. Cl. 800/3; 800/9

(57) ABSTRACT

The present invention relates the function of Dickkopf 3 (Dkk3 ) in testis using shRNA mediated knock down model. Specifically, the present invention provides knock down model comprising reduction in Dkk3 activity. The knock down model of Dkk3 consisting of non human vertebrates which have, incorporated in their genome, shRNA construct targeting mammalian Dkk3 gene exhibits low testis weight, low sperm count and has low litter size. The knock down model displays disrupted seminiferous tubules and are subfertile. The present invention model has a role in sex determination as its interruption leads to sex reversal of XY gonads, converting males to females. Sex reversal role of Dkk3 knock down model can find its utility in agricultural applications. The present invention describes Dkk3 as a dual function protein which is associated with sex determination as well as is essential for the process of spermatogenesis. Such testicular functional studies are useful as a model for various disease states of infertility or subfertility and for identifying a potential treatment to overcome idiopathic infertility.

### FIELD OF THE INVENTION

The present invention relates to shRNA mediated knock down of Dkk3 gene in non human vertebrates to investigate and establish the role of Dkk3 gene in spermatogenesis and sex reversal. More specifically, the present invention relates to a knock down model of Dkk3 consisting of non human vertebrates which have, incorporated in their genome, shRNA construct targeting mammalian Dkk3 thereby generating Dkk3 knock down model.

### BACKGROUND OF THE INVENTION

Dickkopf (Dkk) genes comprise an evolutionary conserved small gene family of four members (Dkk 1-4) and a unique Dkk3-related gene, DkkL1 (soggy). They encode secreted proteins that typically antagonize wingless-related MMTV integration site (Wnt)/beta-catenin signaling, by inhibiting the Wnt coreceptors Lrp5 and 6 (Zorn A. M., 2001). Wnts are intercellular growth and differentiation factors that regulate several key developmental steps, such as gastrulation, neurulation, and organogenesis, including the development of the midbrain, central nervous system, kidney, and limbs (Heikkila et al., 2001). Wnts are also needed for a normal development of the reproductive system. Deficiency of Wnt-4, -5a, and -7a, for example are known to result in sex reversal, infertility, and/or malformation of the internal and external genitals (Heikkila et al., 2001). Many Wnt ligands that signal via the canonical β-catenin pathway, are expressed in fetal gonads. β-catenin, a key transcriptional regulator of the canonical Wnt pathway and an element of the cell adhesion complex, is essential for various aspects of embryogenesis. (Liu C. F. et al, 2009). Role of β-catenin and Wnt molecule have been shown in sex determination. Wnt-4 signaling has been implicated in female development, because its absence leads to partial female to male sex reversal in the mouse (Heikkilä et al 2005).

Dkks (group of proteins) play an important role in vertebrate development, where they locally inhibit Wnt regulated processes such as antero-posterior axial patterning, limb development, somitogenesis and eye formation. Dkk proteins have been implicated in various diseases, including retinal degeneration (Hackam et al. 2004), malignancies (Hsieh et al. 2004), Alzheimer's disease (Alvarez et al. 2004) and cerebral ischemia (Mastroiacovo et al. 2009). Dkk3 is expressed during vertebrate development in many organs (Del Barco Barrantes et al., 2006, Monaghan A. P, et al 1999). Prominent expression of Dkk3 is observed in the brain and in fibroblasts of adult rodents (Hackam A S et al., 2004). It is also found in the human adrenal cortex (Suwa T et al., 2003). Dkk3 has been proposed to act as a tumor suppressor, as it is down regulated in a number of tumor cells and Dkk3 over expression suppresses cell growth (Hsieh S. Y et al., 2004). Hence, Dkk3 is also known as REIC (for reduced expression in immortalized cells) (Tsuji T et al., 2000). Dkk3 has been found to be frequently inactivated in lung cancer. Del Barco Barrantes (2006) described Dkk3-deficient mice as euthyroid. Altered phenotypes in Dkk3 mutant mice were observed in the frequency of NK cells, immunoglobulin M, hemoglobin, and hematocrit levels, as well as lung ventilation. So far the biological properties of Dkk3 protein have not been evaluated in testis; therefore we studied the role of Dkk3 gene in testis. Worldwide, human infertility affects 10-15% of couples of which approximately 30-50% is attributable to male infertility. 70-90% of which arises from disrupted or impaired spermatogenesis with a clinical outcome of azoo- or oligospermia (Hull A. G et al., 1985). At present, treatments for male infertility are limited and most often a range of assisted reproduction techniques are used to circumvent rather than treating male infertility problems permanently. Several environmental, behavioural and genetic factors have been known to affect male fertility. However, there is very little information available on the mechanism of such effects. To develop true therapies, one would require a deeper understanding of the genes involved in regulation of spermatogenesis. Under these circumstances, we evaluated the role of this specific gene Dkk3 in process of spermatogenesis in gene knock down model. This study shows the testicular function of Dkk3. To interfere with the function of Dkk3 gene, the knock down model was generated using shRNA construct targeting Dkk3. Such knock down models are useful to study various disease states of infertility or subfertility and for identifying agents that can act as potential therapeutic agent.

### SUMMARY OF THE INVENTION

The present invention evaluates the role of Dkk3 in testis in regulation of spermatogenesis and sex determination by generating a knock down model which have, incorporated in their genome, shRNA construct targeting mammalian Dkk3 mRNA. To interfere with the function of Dkk3 gene, the knock down model was generated using shRNA constructs targeting Dkk3. It provides the testicular role of Dkk3 by generating knock down model system of Dkk3 gene which will provide a novel tool for the study of spermatogenesis. The present invention establishes Dkk3 as a dual function protein associated with sex determination during embryogenesis, where Dkk3 directs bipotential gonads towards male phenotype naturally. In fact, the present invention establishes that knock down of Dkk3 lead to sex reversal of XY gonads resulting into females. During adulthood, a higher level of Dkk3 is required for the maintenance of spermatogonial stem cell division and differentiation as knocking down of Dkk3 lead to disruption in spermatogenesis varying from oligozoospermia to Azoospermia, resulting in subfertility as well as infertility.

Accordingly, the main objective of this invention is to determine the role of Dkk3 in testis in the regulation of spermatogenesis and in sex determination by generating a knock down model which have, incorporated in their genome shRNA construct, which targets mammalian Dkk3 mRNA.

The present invention, therefore, evaluates the role of Dkk3 in testis during embryogenesis and adult stage of development by creating a knock down model of non human vertebrates, which have incorporated in their genome shRNA construct targeting mammalian Dkk3 gene. Dkk3 has dual function, the protein is associated with sex determination during embryogenesis, where Dkk3 directs bipotential gonad towards male pathway and it is also necessary for the process of spermatogenesis.

Another embodiment of the present invention envisages a Dkk3 knock down model for assessing the role of the gene in the testis. Since it utilizes cytomegalovirus (CMV) promoter, function of Dkk3 can be analyzed in any other organ as well e.g. liver, kidney, brain, lungs etc.

Fertility problems affect 10% of couples in our society either due to genetic or environmental causes, making it one of the most common of serious health issues. Despite this, little is known about the various causes of infertility. With infertility being such a common problem, identification of any cause would impact on a large number of patients.

The present invention provides methods to identify a cause of infertility in particular, by evaluating the function of the Dkk3 gene in maintenance of male reproductive function.

The present invention relates generally to a non human vertebrate model useful in a method for the treatment of infertility or reduced fertility in males. The aspect of the present invention is to provide a strong factor for the screening of infertility and for elucidating the mechanisms involved in the process of spermatogenesis. This can be done by identifying a hitherto unknown function of Dkk3 gene in testis and by developing a model having reduced Dkk3 activity and analyzing its effect on fertility.

The present invention contemplates a method for the treatment of infertility or reduced fertility in a subject or even more particularly a method of modulating spermatogenesis in a non human vertebrate associated with Dkk3. Such non-human vertebrates fail to undergo productive spermatogenesis and can be used as a model to screen for therapeutic molecules capable of inducing, enhancing or otherwise facilitating spermatogenesis in Dkk3 knock down non-human vertebrates.

Treatments that may potentially cure the disease or relieve its symptoms may be tested first in a Dkk3 knock down model which exhibits infertility/subfertility by administering the potential treatment to non human vertebrate and observing the effects, and comparing the treated Dkk3 knock down non human vertebrate to untreated controls. Such aspects of the present invention may find utility of Dkk3 knock down in male contraception.

The model may also be used to study aspects of sex reversal.

Moreover, due to the high degree of homology between the human and mouse Dkk3 gene, shRNA can be targeted against Dkk3 in order to induce infertility in any species as a form of animal husbandry.

Knocking down of Dkk3 leads to sex reversal phenomena with males converting to females. Such aspect of the present invention may find its utility in agriculture where adult female cattle are reproduced and bred to increase milk production and to increase the production of dairy products.

Techniques like embryo transfer, somatic cloning to particularly produce female cattle are quite cumbersome, expensive and require technical expertise. The present invention provides Dkk3 as a target gene for male-to-female sex reversal. Knocking down of Dkk3 as a tool to generate more female cattle is technically superior, less expensive, less cumbersome and less time consuming.

The specific role of Dkk3 in male-to-female sex reversal makes it a potential candidate in agricultural applications, where preferential use of females over males generates a requirement for sex sorting.

### BRIEF DESCRIPTION OF DRAWINGS

The invention will now be described in greater detail with reference to the accompanying drawings wherein:

FIG. 1(a)-1(b) depict a) the PCR results of genomic DNA (gDNA) of F1 progeny generated from the mating of electroporated male and wild type female.
Lane 1: 100 bp marker
Lane 2: gDNA of wild type, Lane 3: Blank, Lane 4-12: gDNA of Dkk3 knock down animals, Lane 13: plasmid DNA
b) Slot-blot analysis for genomic integration of shRNA construct in Dkk3 knock down animals.
Samples 1-14-Dkk3 knock down animals gDNA, Sample 15-Wild type gDNA.

FIG. 2(a)-2(b) depict a) the quantitative real time PCR analysis of Dkk3 mRNA levels in testes of Dkk3 shRNA knock down model relative to wild type testes at ten weeks of age (n=3).
b) the western blot analysis of Dkk3 protein levels from the testes of Dkk3 shRNA knock down model and wild type. Lane 1-2: Testes from two different wild types. Lane 3-5: Testes from three different Dkk3 shRNA knock down models.

FIG. 3(a)-3(d) depict the immunohistochemical localization of GFP expression in the testis of Dkk3 shRNA knock down model. GFP antibody staining of bouin's fixed, paraffin embedded testicular sections. a) and b) Fluorescence and merged image of the testicular sections of Wild type. c) And d) Fluorescence and merged image of the testicular sections of Dkk3 knock down model.

FIG. 4(a)-4(d) depict the immunohistochemical localization of Dkk3 protein in testicular sections of wild type and Dkk3 shRNA knock down model. a) and b): Fluorescence and merged image of the seminiferous tubules of wild type model. c) and d) Fluorescence and merged image of the seminiferous tubules of Dkk3 shRNA knock down model. Very few cells stained positive for Dkk3 .

FIG. 5(a)-5(d) depict a) the graphical representation of mean testis weight (in mgs) of wild type and Dkk3 shRNA knock down models at ten weeks of age. (n=10).
b) the graphical representation of mean sperm count (million/ml) from epididymis of wild type and Dkk3 shRNA knock down model at ten weeks of age. (n=10).
c) the graphical representation of litter size of wild type matings and Dkk3 shRNA knock down model matings (n=3).
d) the serum testosterone levels of wild type and Dkk3 shRNA knock down model before and after testosterone replacement. Number of asterisks represents degree of statistical significance (n=3).

FIG. 6(a)-6(d) depict the testicular histology of wild type and Dkk3 shRNA knock down model showing varying degree of abnormalities in seminiferous tubules (20× magnification)
a) Seminiferous tubules of wild type and Dkk3 model at ten weeks of age.
b) Seminiferous tubules of wild type and Dkk3 model at one year of age.

FIG. 7 depicts the quantitative real time PCR analysis of Wnt genes mRNA levels in testes of Dkk3 shRNA knock down model relative to wild type testes at ten weeks of age. Number of asterisks represents degree of statistical significance (p<0.05) (n=3).

FIG. 8(a)-(d) depict the immunohistochemical localization of Mullerian inhibiting substance (MIS) protein in testicular sections of wild type and Dkk3 shRNA knock down model.
a) and b): Fluorescence and merged image of the seminiferous tubules of wild type model. c) and d) Fluorescence and merged image of the seminiferous tubules of Dkk3 shRNA knock down model.

FIG. 9(a)-(d) depicts the immunohistochemical localization of Glial derived neurotrophic factor (GDNF) protein in testicular sections of wild type and Dkk3 shRNA knock down model. a) and b): Fluorescence and merged image of the seminiferous tubules of wild type model. c) and d) Fluorescence and merged image of the seminiferous tubules of Dkk3 shRNA knock down model.

FIG. 10(a)-10(b) depict a) the SRY gene sex genotyping.
Lane 1: 100 bp marker, Lane 2-4: Blank
Lane 5-7: gDNA of wild type females (negative control), Lane 8: gDNA of wild type male (positive control), Lane 9-10: gDNA of wild type males (positive control)
Lane 11-16: gDNA of Dkk3 knock down females, Lane 17: Dkk3 shRNA plasmid (negative control)
b) Shows the SRY gene sex genotyping.
Lane 1: 100 bp marker, Lane 2-4: Blank
Lane 5: gDNA of wild type females (negative control),
Lane 6-13: gDNA of Dkk3 knock down females, Lane 14: gDNA of wild type male (positive control)

### DETAILED DESCRIPTION OF THE INVENTION

The invention provides knock down models of Dkk3 and relates the role of Dkk3 gene in regulation of process of spermatogenesis and sex determination.

Prior to setting forth the invention in detail, it may be helpful to define the following terms and/or expressions in the context they are used in the present invention. The definition of the expressions provided hereafter is non-limiting in nature.

Testes—The male sex gland in the scrotum in which sperm and testosterone are produced. There is a pair of testes behind the penis in a pouch of skin called the scrotum. The testes make and store sperm, and make the male hormone testosterone.

Non-Human Vertebrates includes all vertebrates except human beings like guinea pig, rabbit, rodents, dog etc.

DNA—Deoxyribonucleic acid (DNA) it constitutes the primary genetic material of all cellular material, it occurs predominantly in the nucleus.

Spermatogenesis—This process includes all of the nuclear and cytoplasmic changes that transform the primordial germ cells of the male germ line into mature spermatocytes. The formation of mature sperm in the male testes after the onset of puberty.

Seminiferous Tubules—Seminiferous tubules are located in the testes, and are the specific location of meiosis, and the subsequent creation of gametes, namely spermatozoa. These are the tubules lining in the testes that produce sperm.

Genes—A length of DNA that carries the genetic information necessary for production of a protein. Genes are located on chromosomes and are the basic units of heredity.

Promoter—It is a controlling element in the expression of the gene. It serves as a recognition signal for an RNA polymerase and marks the site of initiation of transcription. A promoter is a region of DNA that facilitates the transcription of a particular gene.

Knock down—Suppression of the expression of a gene product, typically achieved by the use of antisense oligo deoxynucleotides and RNAi that specifically target the RNA product of the gene. Gene knock down refers to techniques by which the expression of one or more of an organism's genes is reduced, either through genetic modification (a change in the DNA of one of the organism's chromosomes) or by treatment with a reagent such as a short DNA or RNA oligonucleotide with a sequence complementary to either an mRNA transcript or a gene. If genetic modification of DNA is done, the result is a “knock down organism”.

shRNA—Small hairpin (shRNA) contains sense and antisense sequences from a target gene connected by a loop, and is expressed in mammalian cells from a vector by a pol III-type promoter. The shRNA is transported from the nucleus into the cytoplasm, where Dicer processes it. Small hairpin RNA is expressed from a DNA template and processed into small RNAs to guide RNAi-mediated targeted mRNA degradation.

shRNA vector—Small hairpin RNA (shRNA) vector is a DNA molecule used as a vehicle to transfer foreign genetic material into another cell. ShRNA vector can express shRNA with the help of its promoter.

Wild type—the normal, typical phenotype of any mammal before genetic mutation takes place. Wild type refers to the phenotype of the typical form of a species as it occurs in nature.

Electroporation—The application of electric current to a living surface (as the skin or plasma membrane of a cell) in order to open pores or channels in cells or tissue through which a biological material (a drug or DNA) may pass. It is the use of electrical pulses to enable cells to take up DNA.

F1 Generation—F1 Generation is produced by mating of shRNA knock down male with the wild type female.

Primer—A primer is a strand of nucleic acid that serves as a starting point for DNA replication.

Real time PCR—real-time polymerase chain reaction, also called quantitative real time polymerase chain reaction, is a laboratory technique based on the PCR, which is used to amplify and simultaneously quantify a targeted DNA molecule. It enables both detection and quantification (as absolute number of copies or relative amount when normalized to DNA input or additional normalizing genes) of one or more specific sequences in a DNA sample.

Antibody—Antibodies also known as immunoglobulins are gamma globulin proteins that are found in blood or other bodily fluids of vertebrates, and are used by the immune system to identify and neutralize foreign objects, such as bacteria and viruses. Molecules produced by B cells in response to specific proteins (antigens) carried by infected cells.

Progeny—New individual organisms that results from the process of sexual or asexual reproduction.

Wnt Pathway—The Wnt pathway involves a large number of proteins that can regulate the production of Wnt signaling molecules, their interactions with receptors on target cells and the physiological responses of target cells that result from the exposure of cells to the extracellular Wnt ligands. It describes a network of proteins best known for their roles in embryogenesis and cancer, but also involved in normal physiological processes like reproduction in adult animals.

Expression—Process by which polynucleic acids are transcribed into mRNA and translated into peptides, polypeptides, or proteins.

One of the preferred embodiments of the present invention resides in a process comprising the following steps:
1. Dkk3 specific shRNA sequences are synthesized and cloned into pRNAT-CMV3.1/Neo vector (GenScript USA Inc.). Positive clones may be confirmed by sequencing. shRNA clones can be linearized with Sal I and 4 kb DNA fragment may be eluted.
2. Generation of Dkk3 shRNA knock down model by electroporation of linearized cloned shRNA construct into testis.
3. Integrated shRNA construct may be detected by several means well known to those skilled in the art. Non limiting examples include PCR, slot-blot, where gDNA sample may be analyzed for integration of shRNA.
4. Another method of detection used is quantitative real time PCR that may be done to investigate the relative presence of the transcribed RNA from the target gene as a result of shRNA knocking down transcribed mRNA. This is also a measure of its efficacy.
5. Other non limiting illustrative examples of detection include Western blot analysis that can be done using an antibody against the protein encoded by the gene. These methods may be employed as alternative or additional methods for evaluating the knockdown of the gene.
6. Since the target gene plays a role in spermatogenesis, the reproductive status of the shRNA knock down model may be assessed by taking testis weight, sperm count and fertility test.
7. Testicular tissue may also be analyzed directly, for example, by preparing tissue sections.

In some embodiments, it may be preferable to fix the tissue immersing in Bouin's solution. Tissue section can be paraffin embedded. Slides of the tissue may be used for immunohistochemistry or histological staining with eosin and hematoxylin.
8. To ascertain the mechanism of action, downstream signaling intermediates can also be assessed by real time PCR, western blot and immunohistochemistry.

For the purposes of greater illustration, the invention will be described non-limitatively with reference to the accompanying drawings in the following examples which are not limited to sub-human primates such as, guinea pigs, rabbits, rats, mice, dogs, etc.

### Example 1

### Generation of Dkk3 Knock Down Model

The shRNA constructs can be designed with the aim of knocking down specific gene expression. Dkk3 specific shRNA sequences may be synthesized and cloned into pRNAT-CMV3.1/Neo vector (GenScript USA Inc.). The shRNA vector comprises of CMV promoter which drives the expression of shRNA and a SV40 promoter drives the expression of the GFP. This vector carries GFP for convenient tracking. Dkk3 specific shRNA cassettes can be easily inserted into the vector between BamHI and AflII sites. Positive clones may be confirmed by sequencing. shRNA clones can be linearized with Sal I and 4 Kb DNA fragment can be eluted. shRNA sequences for the knock down of Dkk3 gene are mentioned in Table 1.

TABLE 1
shRNA sequences for knock down of Dkk3

| GENE | |
|---|---|
| | FORWARD OLIGO |
| Dkk3 | GATCGTACCAATTGGCAGGAAGTTCACAAGATAACCAATCA |
| | AGAGTTGGTTATCTTGTGAACTTCCTGTTTTTTCAATTGGTAC |
| | REVERSE OLIGO |
| Dkk3 | TTAAGTACCAATTGAAAAAACAGGAAGTTCACAAGATAACC |
| | AACTCTTGATTGGTTATCTTGTGAACTTCCTGCCAATTGGTAC |

Eluted linearized shRNA construct having Dkk3 shRNA is used for in vivo electroporation (Dhup and Majumdar 2008). A non human vertebrate male may be used for shRNA construct electroporation in testis. Two males per shRNA construct may be electroporated. Thirty five days post electroporation, each electroporated male can be cohabitated with two wild type females for natural mating. Offspring born may be analyzed for shRNA integration by isolating genomic DNA.

### Example 2

### Genotyping by Polymerase Chain Reaction and Slot-Blot

A tissue from offspring sired by electroporated males are taken and lysed for 16 hours at 55° C. in high salt digestion buffer containing 50 mM Tris HCl, 1% SDS, 100 mM NaCl, 100 mM EDTA and 1200 μg/ml Proteinase K. The lysate can be processed for isolation of DNA using phenol-chloroform extraction followed by ethanol precipitation. Extracted genomic DNA is subjected to PCR analysis using primers. Primers are designed with forward primer on SV40 promoter and reverse primer on GFP gene, so that knock down model generated against Dkk3 specific shRNA may be screened (Table 2). Every PCR reaction set has two controls. PCR of shRNA construct plasmid is used as a positive control, and PCR of gDNA obtained from wild type is used as a negative control. The PCR reaction is performed using Perkin Elmer Thermal Cycler. Reaction conditions are as follows: 94° C. for 5 minutes followed by 30 cycles of 94° C. for 30 seconds, 60° C. for 30 seconds and 72° C. for 30 seconds. The gDNA of offspring is screened for the presence of the Dkk3 shRNA by PCR. Presence of a 500 bp product in PCR confirmed genomic integration of the construct in shRNA knock down model (FIG. 1a).

TABLE 2
Primers used for genomic PCR analysis

| | Primer sequences |
|---|---|
| | 5′→ 3′ |
| Forward | GCCCCATGGCTGACTAATTT |
| Reverse | GTATCGCCCTCGAACTTCAC |

Slot-blot hybridization with Dkk3 shRNA specific probe confirmed for genomic integration of shRNA in Dkk3 knock down model (FIG. 1b). Slot-blot analysis can be done with gDNA. 1 μg gDNA samples may be slot blotted onto a nitrocellulose membrane using a minifold I apparatus. Pre-hybridization, hybridization with probe and washings may be done following standard procedure. Kodak biomax film can be exposed at −70° C. for 48 hrs. The probe used is 4 kb SalI fragment of pRNAT-CMV3.1 Neo vector containing Dkk3 shRNA sequence.

### Example 3

### Quantitative Real Time PCR Analyses

To confirm gene knock down and measure its efficacy, the relative expression of gene in testis of Dkk3 shRNA knock down model may be investigated by quantitative real time PCR. Downstream signaling Wnt pathway molecules may be assessed by quantitative real time PCR. Testes of shRNA knock down model can be snap-frozen, tissue can be ground in pestle-mortar and stored in Trizol (Sigma chemical Co., USA) at −80° C. RNA may be isolated from Trizol treated samples using manufacturer's instructions. Real time PCR can be done using different primers specific for respective genes (Table 3). 0.5 μg of RNA was treated with Dnase I (0.5 μg) for 15 minutes at 25° C. Reaction may be terminated by adding 1 μl of 25 mM EDTA and incubating at 65° C. for 10 minutes. RNA can be reverse transcribed using Promega kit following manufacturer's instructions. Real time PCR may be performed using 1 μl of cDNA in a reaction volume of 10 μl. SYBR green can be used from Applied Biosystem. Knock down model showed up to 96% reduction in Dkk3 expression levels compared to wild type controls levels (FIG. 2a).

TABLE 3
Primers used for Real time PCR analysis of
gene expression

| | Forward Primer | Reverse Primer |
|---|---|---|
| cDNA | Sequence 5′ to 3′ | Sequence 5′ to 3′ |
| Dkk3 | TCCCTTTCTGGCTAACAGGA | ACCAAAGCTGCAGAAGTCTC |
| Wnt4 | ACTGGACTCCCTCCCTGTCT | TGAGAAGGCTACGCCATAGG |
| Wnt1 | GCAAGGCCAGGCAGGCCATG | CACTCACGCTGTGCAGGATC |
| Wnt3a | CGATGGCTCCTCTCGGATAC | TGCTGACGGTGGTGCAGTTC |
| Wnt8a | GCAGGACCATGGGACACTTG | GAAGGATGTCTCTCTCGTGG |
| Wnt5a | GGAAGGTGGGCGATGCCCTC | TGCAATGACAGCGTTCGGTC |
| Wnt5b | GCAAGGTGGGGGACCGTTTG | CACCTGAACGCTCTTGAAGC |
| Wnt6 | ACGGCTGCTGGAGCGCTTCC | TCTCCTCGAGCTGTACGCTC |
| Wnt11 | CTGACCTCAAGACCCGCTAC | CCACCACTCTGTCCGTGTAG |

### Example 4

### Protein Extraction and Western Blot Analysis of Dkk3 Protein Levels in Wild Type and shRNA Knock Down Model

Testes samples from two different wild type and three different Dkk3 shRNA knockdown models may be lysed with ice-cold PBS (pH 7.4) containing 50 mM Tris Chloride, 150 mM NaCl, 1% Triton X100 and protease inhibitors (1 mm PMSF, 1 μg/ml aprotinin and 1 μg/ml leupeptin) and lysates may be centrifuged (13,000×g, 4° C., 30 minutes). 20 μg protein from supernatant may be resolved by SDS-PAGE and transferred to a nitrocellulose membrane. After blocking, the membranes may be incubated with rabbit anti-mouse Dkk3 antibody at 4° C. for overnight and then with the goat anti-rabbit horseradish peroxidase-conjugated secondary antibody (1:5000) for 2 hours at room temperature. The blots can be developed using chemiluminescence agent (Amersham Biosciences). Testicular samples from shRNA knock down model and wild type showed 38 kDa Dkk3 protein. However, the expression level of protein was lower in shRNA knock down model as compared to wild type. GAPDH was also assessed by western blotting as loading control (FIG. 2b).

### Example 5

### Immunohistochemical Localization in Wild Type and shRNA Knock Down Model

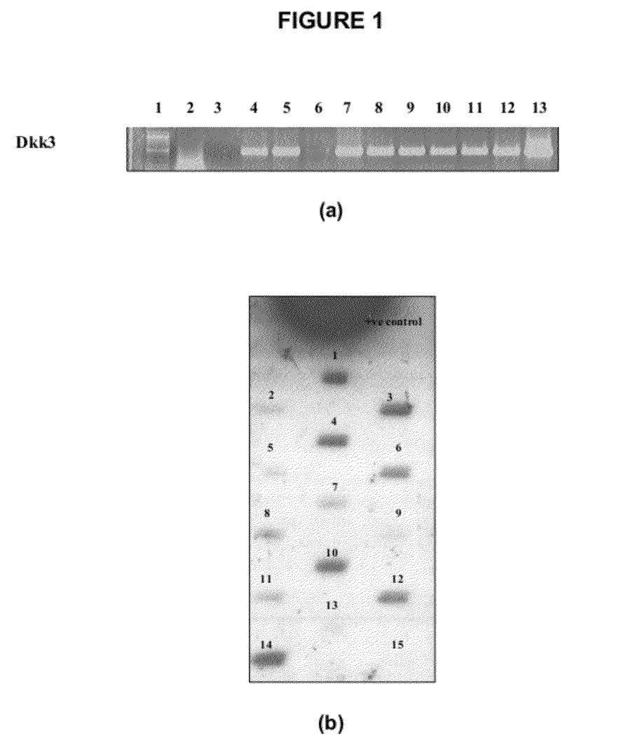

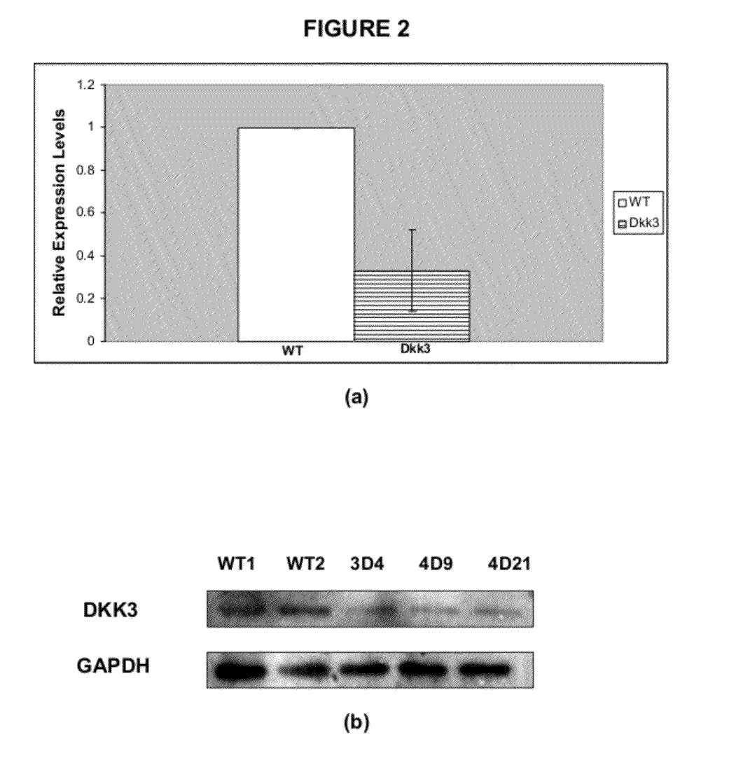

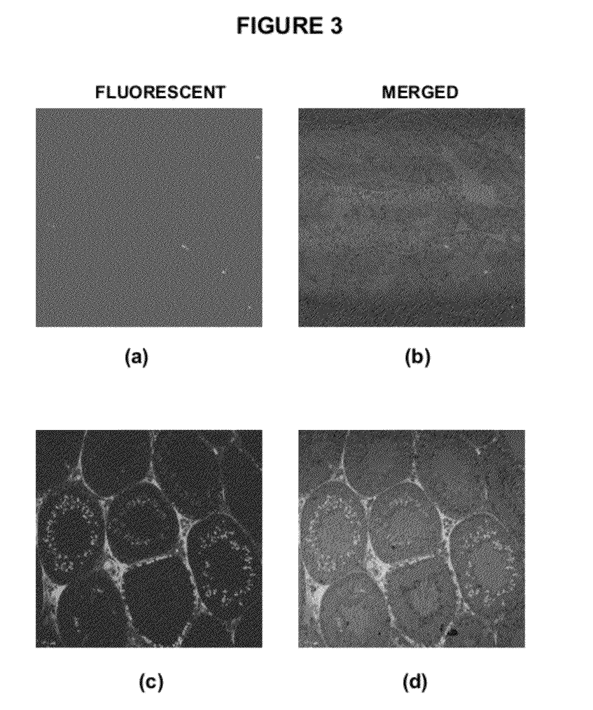

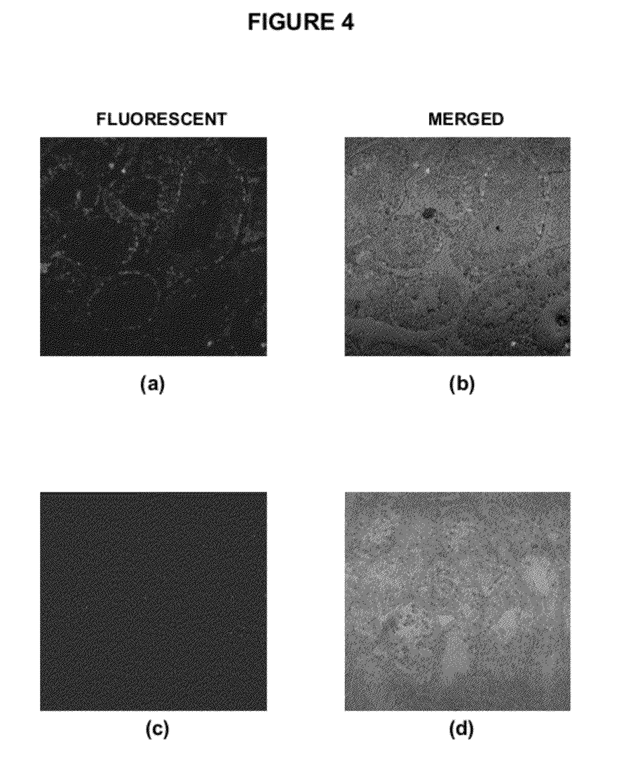

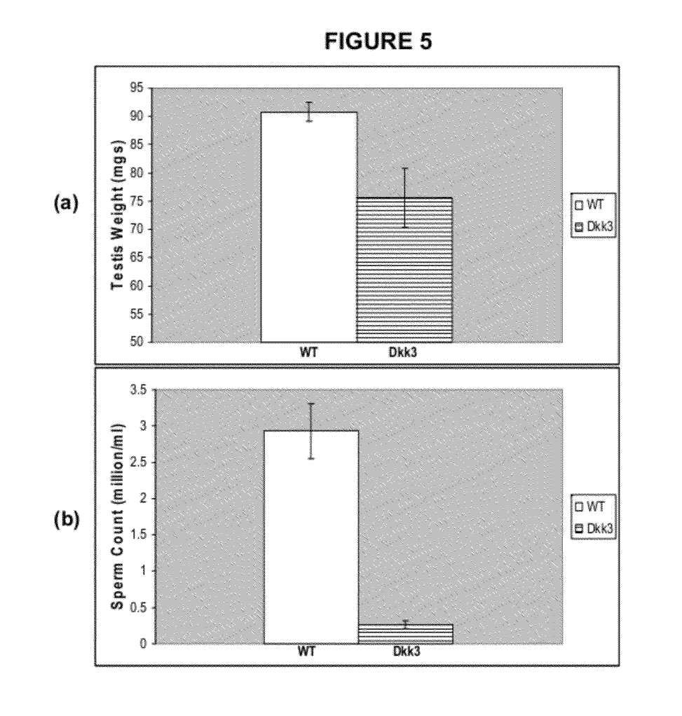

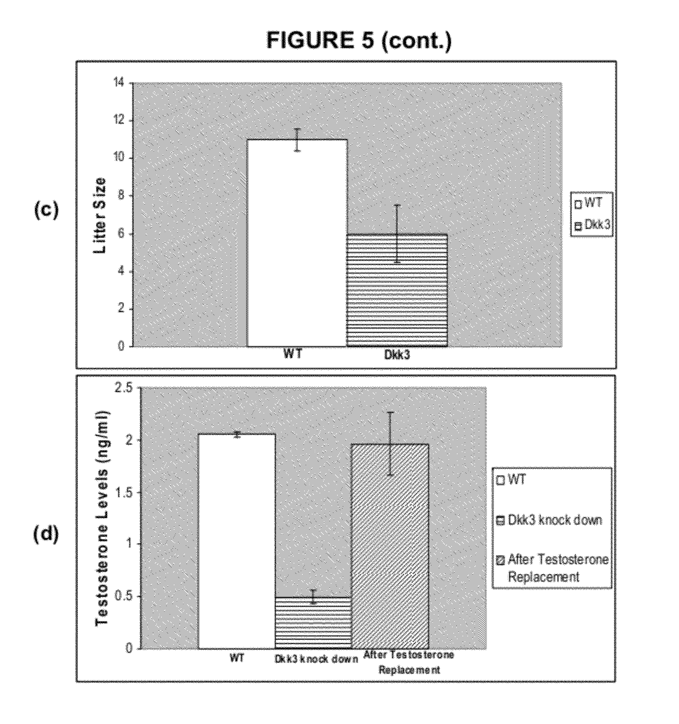

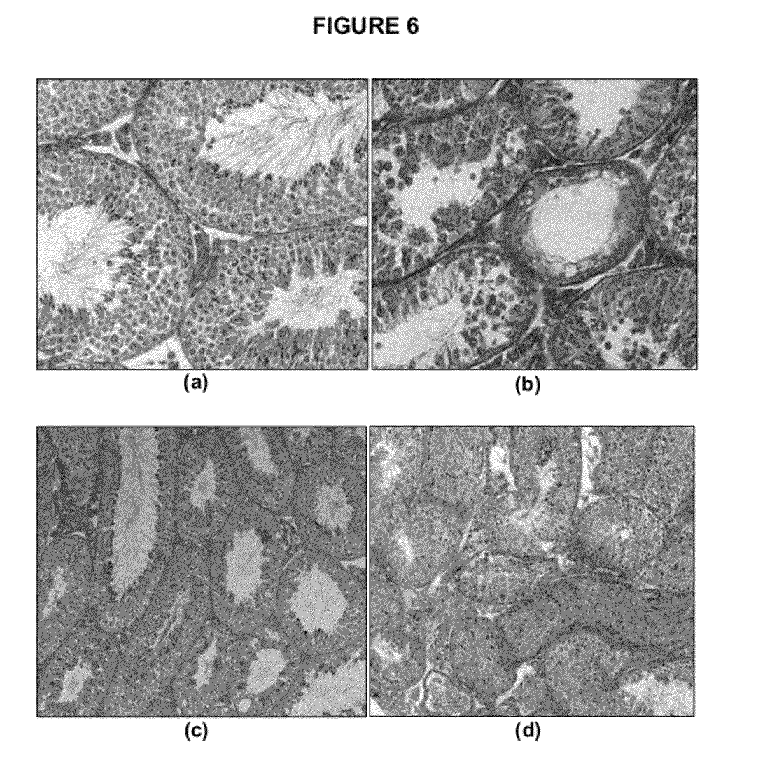

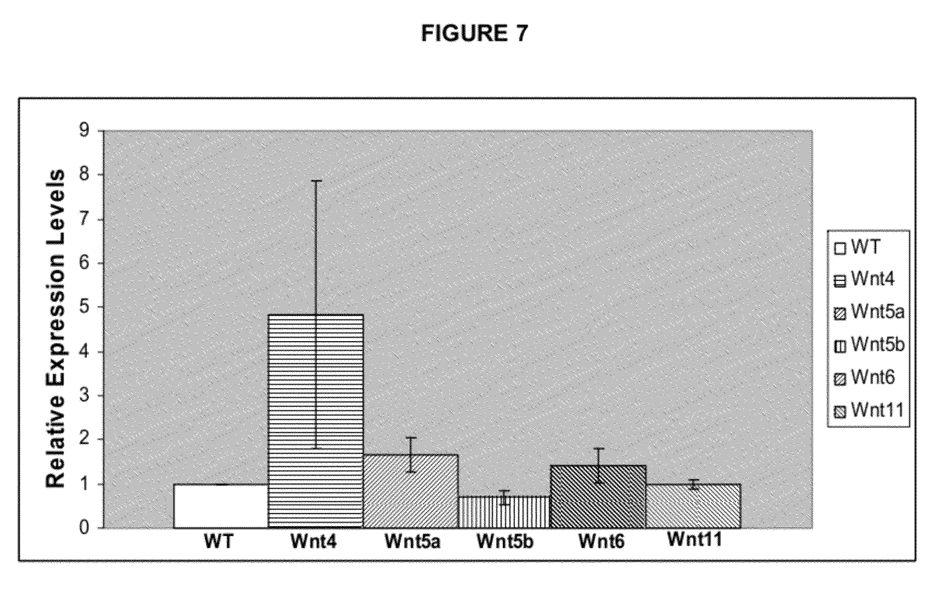

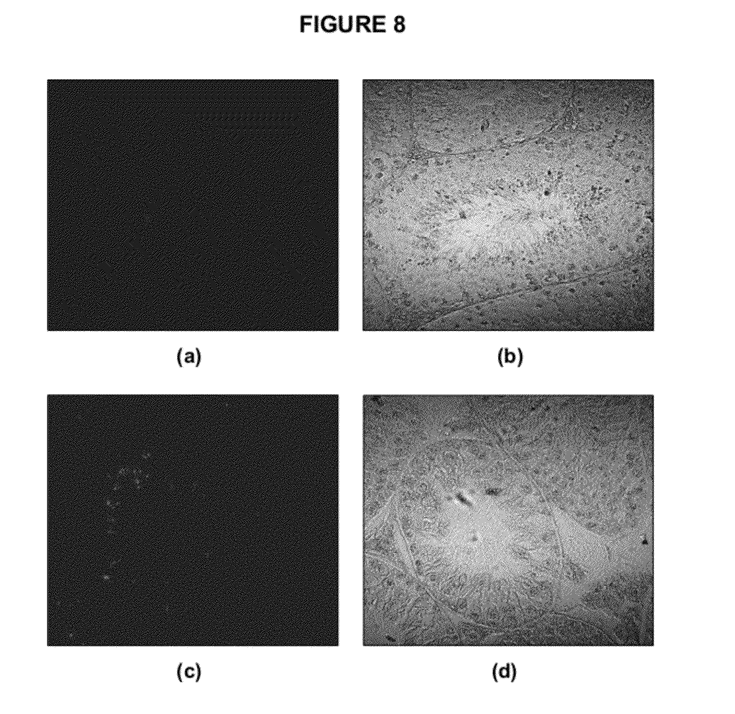

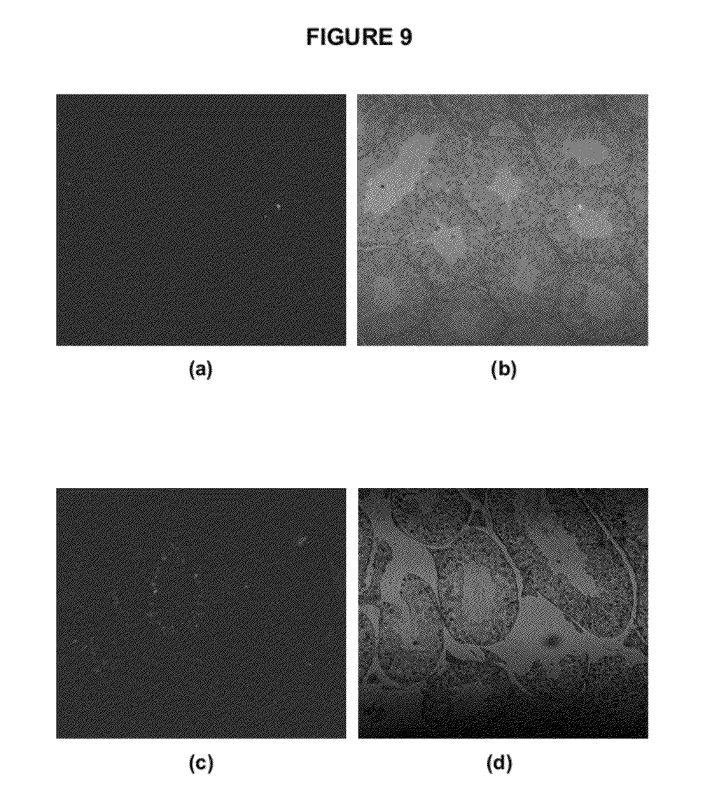

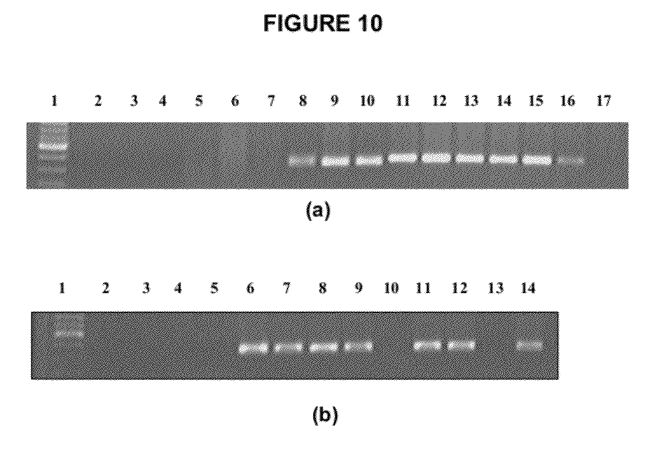

Immunohistochemistry may be performed using Bouin's fixed and paraffin-embedded testicular sections of wild type and F1 generation of shRNA knock down model of Dkk3. Testicular sections can be deparaffinized, rehydrated by successive series of ethanol and rinsed in distilled water. Antigen retrieval may be done and the samples can be blocked using goat serum, permeabilized, and then incubated with standardized dilution of a primary antibody (mouse GFP antibody, rabbit anti-mouse Dkk3 antibody, goat anti-mouse MIS antibody, rabbit anti-mouse GDNF antibody) for overnight at 4° C. Later, after PBS washings, sections may be incubated with standardized dilution of anti-mouse or anti-rabbit Alexa Fluor 488 or anti-goat Cy5 labeled secondary antibody for 4 hours at room temperature in dark. Sections can be analyzed for fluorescence using Olympus IX81 microscope equipped with fluo view SV1000. A specific staining of GFP (FIG. 3), Dkk3 (FIG. 4), MIS (FIG. 8) and GDNF (FIG. 9) may be observed in the testicular sections of shRNA knock down model of Dkk3. Wild type showed no expression of GFP. Dkk3 expression was higher in wild type and diminished in Dkk3 shRNA knock down model. Expression of MIS and GDNF were found to be higher in Dkk3 Knock down model compared to wild type.

### Example 6

### Analyses of Reproductive Potential of Dkk3 Knock Down Model

Testes weight of wild type and Dkk3 knock down males at ten weeks of age may be recorded. At the age of ten weeks, testes of both wild type and Dkk3 knock down model may be weighed. Because of the expression of shRNA, testicular weights of Dkk3 knock down models were significantly less as compared to wild type (FIG. 5a).

To evaluate the biological relevance of the knock down of the gene on sperm production and reproductive capability of knock down model, the concentration and motility of epididymal spermatozoa and the litter size of productive matings may be analyzed. Total numbers of sperms present in epididymus can be counted after releasing the sperms in 1 ml of phosphate-buffered saline by puncturing the epididymus at several sites. The total sperm count may be assessed by using a hemocytometer. We observed that spermatozoa number decreased significantly in epididymal semen of Dkk3 knock down model (0.26±0.05×10⁶/ml/epididymus as compared to wild type (2.93±0.37×10⁶/ml/epididymus) (FIG. 5b).

To evaluate the fertility, male and female knock down model from F1 generation (siblings) may be cohabitated for three weeks. This ensured exposure of female to the male at least through five ovarian cycles. Litter size can be determined as a mean to assess fertility of parent. Similarly, wild types were also assessed for fertility. The total numbers of offspring born from productive matings were recorded. The average litter size was 6.0±1.5 in Dkk3 knock down model which is significantly lower in comparison to wild type where the average litter size was 11.0±0.5 (FIG. 5c).

### Example 7

### Assessment of Serum Hormone Levels and Testosterone Replacement

Serum testosterone concentrations from the Dkk3 knock down model and wild-type can be assessed at 10 weeks of age. Blood can be obtained through retro orbital bleeding before sacrificing the model. Serum testosterone can be assayed using a modified testosterone immunoassay (RIA) system in triplicate. Dkk3 knock down males had significantly lower serum testosterone levels than wild type littermates controls at 3 months of age (FIG. 5d). For testosterone replacement, Dkk3 knock down model can be injected intramuscularly with 5 mg of testosterone undecanoate. Controls may be injected with castor oil. Blood can be taken after one month of the injection and serum testosterone levels may be measured. Histological analysis of Dkk3 knock down model testes did not showed any improvement in the degree of spermatogenesis tubule disruption upon exogenous testosterone treatment as compared to castor oil treated controls. Sperm counts showed marginal increase as compared to Dkk3 knock down model but not up to wild type control levels. Testosterone replacement studies suggested that other factors apart from testosterone decrease, mainly enhanced Wnt signaling by Dkk3 knock down were contributing majorly towards the disturbed state of spermatogenesis in Dkk3 knock down model testes.

### Example 8

### Histological Examinations

The left testes from three months old and one year aged Dkk3 knock down model as well as wild type may be fixed by immersion in Bouin's solution at room temperature for 24 hours. Testes may be dehydrated through an ethanol series, embedded in paraffin wax, sectioned by standard procedures and sections of 4 μm were obtained and stained with hematoxylin and eosin for evaluating the status of spermatogenesis. Stained slides may be examined using bright field microscopy. Testes of three months old Dkk3 knock down model showed many seminiferous tubules with clear signs of degeneration, as evidenced by the presence of multiple vacuoles (FIG. 6a). This degenerative process was observed in most of the tubules. Majority of tubules showed sloughing off of germ cells. Progressive atrophy of the tubules at all stages of the cycle was noted in older animals, leading to the absence of a tubular lumen and depletion of germ cells. Degeneration of elongated spermatids as well as progressive disorganization and loss of round spermatids, spermatocytes, and spermatogonia were also observed and lead to the apparent loss of germ cells. At old age, some seminiferous tubules also featured multilayered, focal accumulations of cells with long, ovoid nuclei; such cells were stacked on top of each other and situated on the basement membrane within the tubule and were not readily identifiable as either germ or Sertoli cells. Degeneration of the seminiferous tubules was accompanied by an apparent hyperplasia of the testicular interstitial cells, which became particularly evident in older models. (FIG. 6b).

### Example 9

### Expression Analysis of Different Wnt Genes

Dkk3 belongs to a family of Wnt antagonists. To study the effect of Dkk3 knock down on Wnt signaling, different canonical and non-canonical Wnts expression may be analyzed by real time PCR in knock down model and age matched controls (as mentioned in example 3 and table 3). Wnt-4 was highly expressed in Dkk3 knock down adult testis compared to controls. Wnt3a was not detected in testis. The expression of the other non-canonical Wnt mRNAs, including Wnt-6, Wnt-11, Wnt-5a, and Wnt-5b and canonical Wnt-1 and Wnt-8a did not varied (FIG. 7).

### Example 10

### Knock Down of Dkk3 Causes Male-to-Female Sex Reversal

To determine whether Dkk3 knock down leading to enhanced Writ signaling can disrupt male development and drive development of XY gonads towards female phenotype, the litters from the matings of Dkk3 knock down models may be analyzed. Litters from this cross yielded approximately equal numbers of XX (female) and XY (male). Sex genotyping of the phenotypically female Dkk3 knock down models may be done by PCR using SRY specific Primers (Table 4). The PCR results revealed the presence of SRY gene (FIGS. 10a and 10b). Most of Dkk3 knock down female models possessing completely feminized external genitalia were SRY positive and were indistinguishable from XX wild type. Internal reproductive tracts of XY females lacked male organs and instead resembled those of females. The sex-reversed gonads were located near the kidneys similar to wild-type ovaries, and reproductive tracts were found to have a uterus and vagina. Mating of such XY females with male siblings produced litter size comparable to SRY negative females. Thus male-to-female sex reversal was occurring owing to the knock down of Dkk3.

TABLE 4
SRY primers used for sex genotyping

| | Primer sequences |
|---|---|
| | 5′→ 3′ |
| Forward | CGTGGTGAGAGGCACAAGT |
| Reverse | GGTGTGCAGCTCTACTCCAG |

Those skilled in the art will recognize or be able to ascertain using no more than routine experimentation, many equivalents to the specific embodiments of the invention described herein. Such equivalents are intended to be encompassed by the following claims.

1. A knock down model of Dickkopf Homologue 3 (Dkk3), wherein said Dkk3 model comprises a non human vertebrate which has incorporated in its genome a shRNA construct targeting mammalian Dkk3 gene, said knock down model of Dkk3 exhibiting one or more of the following characteristics:
a) sub fertility;
b) disrupted seminiferous tubules;
c) low testis weight;
d) low sperm count;
e) low litter size; and
f) male-to-female sex reversal trait.

2. The model of claim 1, wherein the Dkk3 knock down model has reduced levels of Dkk3 protein when compared to the wild type non human vertebrates.

3. (canceled)

4. The model of claim 1, wherein reduced expression of Dkk3 in the Dkk3 knockdown model leads to enhanced Wnt signaling.

5. The model of claim 1, wherein reduced expression of Dkk3 in the Dkk3 knock down model leads to enhanced expression of Mullerian inhibiting Substance (MIS) and Glial derived neurotrophic factor (GDNF).

6. (canceled)

7. (canceled)

8. The model of claim 1, wherein function of Dkk3 can be analyzed in any organ of the body for example liver, kidney, brain, or lungs etc.

9. (canceled)

10. (canceled)

11. The model of claim 1, wherein the Dkk3 shRNA is propagated to progeny by natural mating of a male and female of the same species.

12. The model of claim 1, wherein reduced levels of Dkk3 in the Dkk3 knock down model leads to sex reversal in XY gonads converting males to females.

13. The model of claim 12, wherein said sex reversed converted females are utilized further for milk production and agricultural practices.

14-18. (canceled)

19. A model of infertility for the use of the knock down model of Dkk3 of claim 1 in assessment of any treatment reversing infertility.


SEQUENCE LISTING (24 sequences)

SEQ ID NO: 1 - DNA, 84 bp, Artificial Sequence
  Description of Artificial Sequence Synthetic oligonucleotide
gatcgtaccaattggcaggaagttcacaagataaccaatcaagagttggttatcttgtgaacttcctgttttttcaattggtac

SEQ ID NO: 2 - DNA, 84 bp, Artificial Sequence
  Description of Artificial Sequence Synthetic oligonucleotide
ttaagtaccaattgaaaaaacaggaagttcacaagataaccaactcttgattggttatcttgtgaacttcctgccaattggtac

SEQ ID NO: 3 - DNA, 20 bp, Artificial Sequence
  Description of Artificial Sequence Synthetic primer
gccccatggctgactaattt

SEQ ID NO: 4 - DNA, 20 bp, Artificial Sequence
  Description of Artificial Sequence Synthetic primer
gtatcgccctcgaacttcac

SEQ ID NO: 5 - DNA, 20 bp, Artificial Sequence
  Description of Artificial Sequence Synthetic primer
tccctttctggctaacagga

SEQ ID NO: 6 - DNA, 20 bp, Artificial Sequence
  Description of Artificial Sequence Synthetic primer
accaaagctgcagaagtctc

SEQ ID NO: 7 - DNA, 20 bp, Artificial Sequence
  Description of Artificial Sequence Synthetic primer
actggactccctccctgtct

SEQ ID NO: 8 - DNA, 20 bp, Artificial Sequence
  Description of Artificial Sequence Synthetic primer
tgagaaggctacgccatagg

SEQ ID NO: 9 - DNA, 20 bp, Artificial Sequence
  Description of Artificial Sequence Synthetic primer
gcaaggccaggcaggccatg

SEQ ID NO: 10 - DNA, 20 bp, Artificial Sequence
  Description of Artificial Sequence Synthetic primer
cactcacgctgtgcaggatc

SEQ ID NO: 11 - DNA, 20 bp, Artificial Sequence
  Description of Artificial Sequence Synthetic primer
cgatggctcctctcggatac

SEQ ID NO: 12 - DNA, 20 bp, Artificial Sequence
  Description of Artificial Sequence Synthetic primer
tgctgacggtggtgcagttc

SEQ ID NO: 13 - DNA, 20 bp, Artificial Sequence
  Description of Artificial Sequence Synthetic primer
gcaggaccatgggacacttg

SEQ ID NO: 14 - DNA, 20 bp, Artificial Sequence
  Description of Artificial Sequence Synthetic primer
gaaggatgtctctctcgtgg

SEQ ID NO: 15 - DNA, 20 bp, Artificial Sequence
  Description of Artificial Sequence Synthetic primer
ggaaggtgggcgatgccctc

SEQ ID NO: 16 - DNA, 20 bp, Artificial Sequence
  Description of Artificial Sequence Synthetic primer
tgcaatgacagcgttcggtc

SEQ ID NO: 17 - DNA, 20 bp, Artificial Sequence
  Description of Artificial Sequence Synthetic primer
gcaaggtgggggaccgtttg

SEQ ID NO: 18 - DNA, 20 bp, Artificial Sequence
  Description of Artificial Sequence Synthetic primer
cacctgaacgctcttgaagc

SEQ ID NO: 19 - DNA, 20 bp, Artificial Sequence
  Description of Artificial Sequence Synthetic primer
acggctgctggagcgcttcc

SEQ ID NO: 20 - DNA, 20 bp, Artificial Sequence
  Description of Artificial Sequence Synthetic primer
tctcctcgagctgtacgctc

SEQ ID NO: 21 - DNA, 20 bp, Artificial Sequence
  Description of Artificial Sequence Synthetic primer
ctgacctcaagacccgctac

SEQ ID NO: 22 - DNA, 20 bp, Artificial Sequence
  Description of Artificial Sequence Synthetic primer
ccaccactctgtccgtgtag

SEQ ID NO: 23 - DNA, 19 bp, Artificial Sequence
  Description of Artificial Sequence Synthetic primer
cgtggtgagaggcacaagt

SEQ ID NO: 24 - DNA, 20 bp, Artificial Sequence
  Description of Artificial Sequence Synthetic primer
ggtgtgcagctctactccag